(19) United States
(12) Patent Application Publication
(10) Pub. No.: US 2019/0104785 A1
(43) Pub. Date: Apr. 11, 2019

(54) Padded Hand Restraint

(71) Applicant: Renee Natole, Aurora, CO (US)

(72) Inventor: Renee Natole, Aurora, CO (US)

(21) Appl. No.: 16/118,568

(22) Filed: Aug. 31, 2018

Related U.S. Application Data
(60) Provisional application No. 62/570,836, filed on Oct. 11, 2017.

(51) Int. Cl. A41D 19/015; A41D 19/01; A61F 5/37

(52) U.S. Cl. CPC A41D 19/01582; A41D 19/01; A41D 19/0048; A61F 5/3769; A61F 5/3761

(57) ABSTRACT

A padded hand restraint for securing a hand and wrist to a bed frame. The padded hand restraint includes an annularly shaped padded base with an elongated portion designed to receive a hand and a wrist. A lower strap designed to secure the padded base to an object is attached to a lower surface of the padded base. A wrist strap designed to secure a wrist is disposed on an upper surface. A peripheral padding extends outwardly along a perimeter of the padded base, forming an open section. A secondary strap designed to further secure the wrist is disposed on the elongated portion of the peripheral padding. A mesh netting is additionally located along the annular portion of the padded base, the netting designed to secure over the open section such that the hand is visible while restrained. In this way, a patient can be removably restrained in an efficient manner.

### CROSS REFERENCE TO RELATED APPLICATIONS

This application claims the benefit of U.S. Provisional Application No. 62/570,836 filed on Oct. 11, 2017. The above identified patent application is herein incorporated by reference in its entirety to provide continuity of disclosure.

### BACKGROUND OF THE INVENTION

The present invention relates to hand restraint devices. More specifically, the present invention provides a padded base having an upper surface and a lower surface, wherein the lower surface has a lower strap disposed thereon and the upper surface is configured to receive a hand. A wrist strap is disposed on the upper surface and configured to secure a wrist to the padded base. A peripheral padding extends outward from the upper surface, thereby forming an open portion, wherein a mesh netting and secondary wrist strap disposed along an outer surface of the peripheral padding are configured to removably secure overtop the upper portion, thereby enclosing the hand and wrist.

Many patients who are confused or are suffering from severe injuries require special care, and often require restraints while in a hospital bed. These patients require a greater than usual amount of attention from the nursing staff, as they are prone to attempting to remove their IVs or other equipment, thereby further injuring themselves. Typically, nurses are required to use a standard wrist restraint and mitten to cover the hands of the patient and secure the hands to the bed. However, the mittens applied do not have padding and are uncomfortable for most patients. Unfortunately, when the patient's hand is covered by a typical mitten, an IV is either no longer usable in that hand or it is difficult to see how successfully the IV is inserted into the hand. Additionally, the mitten and wrist restraint are typically separate devices that are not kept together, requiring the nurse to find multiple pieces of equipment to restrain the patient. Further, it is difficult to apply these devices, and additionally difficult to find an appropriate size for the patient. Thus, an improved hand restraint device is desired for allowing an individual, such as a nurse, to more easily and comfortably restrain a patient.

In light of the devices disclosed in the known art, it is submitted that the present invention substantially diverges in design elements from the known art and consequently it is clear that there is a need in the art for an improvement to existing hand restraint devices. In this regard the instant invention substantially fulfills these needs.

### SUMMARY OF THE INVENTION

In view of the foregoing disadvantages inherent in the known types of hand restraints now present in the prior art, the present invention provides a padded hand restraint wherein the same can be utilized for providing convenience for an individual when the hand of a user is restrained to a stationary object, as well as to provide safety and comfort to the restrained user.

It is therefore an object of the present invention to provide a new and improved hand restraint device that has all of the advantages of the prior art and none of the disadvantages. The present system comprises a padded base annularly shaped with an elongated portion along a side of the annular shape, wherein the padded base is configured to receive a hand and a wrist along the elongated portion. The padded base has an upper surface and a lower surface, wherein a lower strap is affixed to the lower surface and configured to removably secure the padded base to a stationary object. A wrist strap is disposed on the upper surface along the elongated portion and is configured to removably secure a wrist to the padded base. A peripheral padding extends upwardly along a perimeter of the padded base, such that an open upper section is thereby formed. A secondary strap is disposed along the elongated portion of the peripheral padding and is configured to further secure the wrist of a user. A mesh netting is additionally disposed along the annular portion of the padded base and is configured to removably secure overtop the open section such that the hand is thereby visible and quickly accessible while restrained. In this way, a nurse can removably restrain a patient in an efficient, safe, and comfortable manner.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1A shows a perspective view of an embodiment of the padded hand restraint with the netting and secondary wrist strap unfastened.

FIG. 1B shows a close-up perspective view of an embodiment of the padded hand restraint with the wrist strap fastened.

FIG. 2A shows a perspective view of an embodiment of the padded hand restraint with the secondary wrist strap fastened.

FIG. 2B shows a perspective view of an embodiment of the padded hand restraint with the secondary wrist strap and netting fastened.

FIG. 3 shows a perspective view of an embodiment of the padded hand restraint in use.

FIG. 4 shows a perspective view of an embodiment of the padded hand restraint with the lower surface of the padded hand restraint affixed to a bed frame.

FIG. 5 shows a perspective view of an embodiment of the padded hand restraint in use affixed to a bed frame.

### DETAILED DESCRIPTION OF THE INVENTION

Reference is made herein to the attached drawings. Like reference numerals are used throughout the drawings to depict like or similar elements of the padded hand restraint. The figures are intended for representative purposes only and should not be considered to be limiting in any respect.

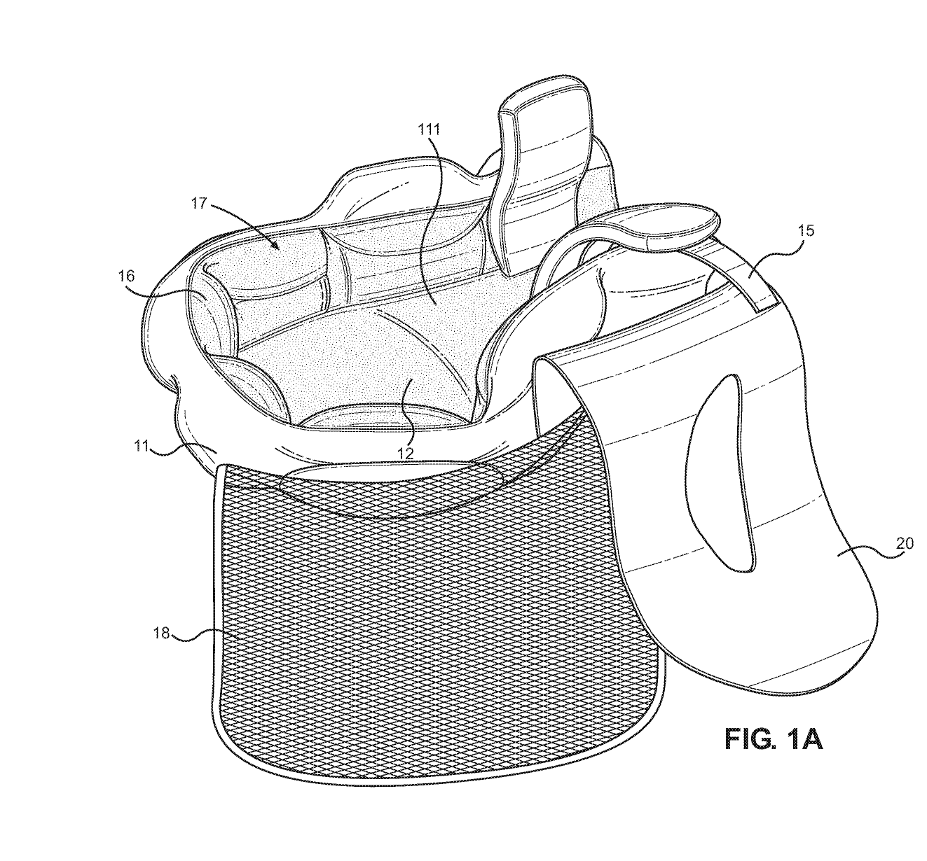

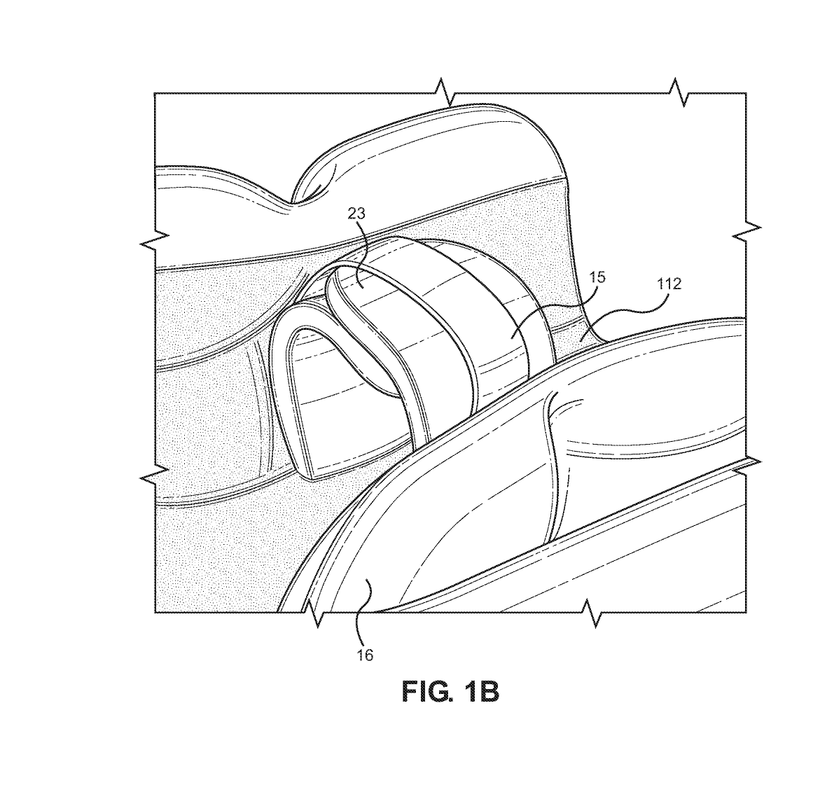

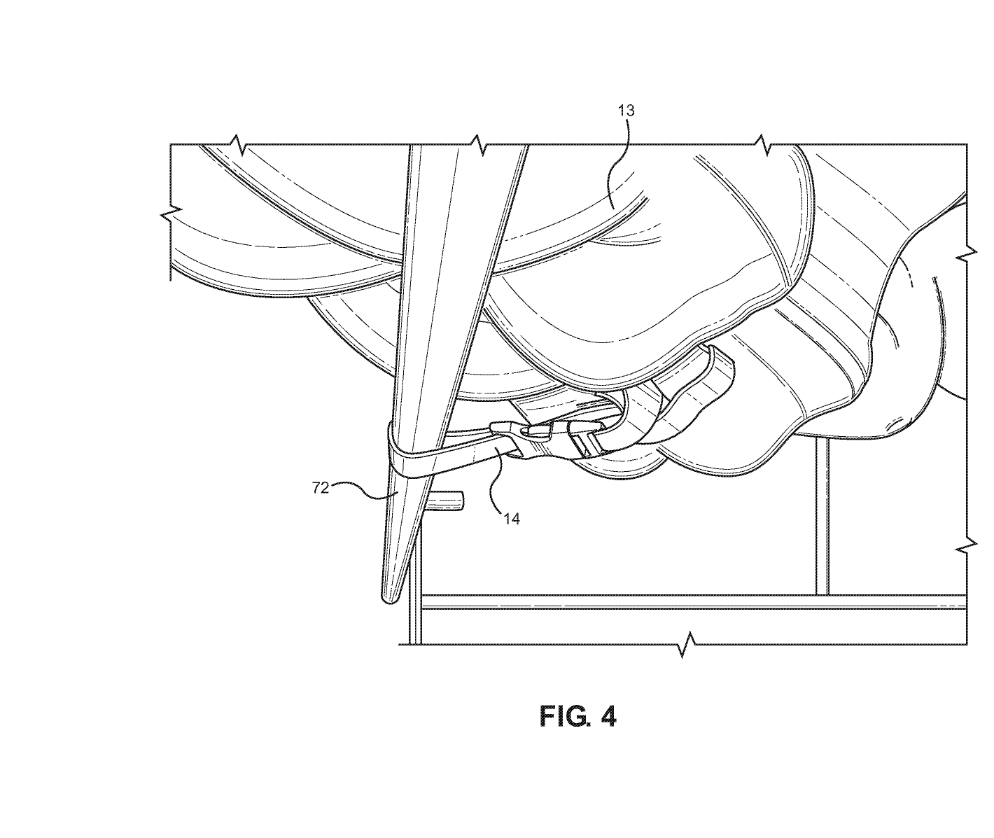

Referring now to FIG. 1A and FIG. 1B, there is shown a perspective view of an embodiment of the padded hand restraint with the netting and secondary wrist strap unfastened, and a close-up perspective view of an embodiment of the padded hand restraint with the wrist strap fastened, respectively. A padded hand restraint comprises a padded base 11 having an upper surface 12 and a lower surface 13 (as seen in FIG. 4). The padded base 11 comprises an annularly shaped portion 111 with a generally straight protrusion 112 along a rear portion of the perimeter, such that a mitten shape is formed. The padded base 11 is configured to receive a wrist along the protrusion 112 and a hand in an open portion 17 defined by the annularly shaped portion 111. In one embodiment, the padded base 11 is composed of a hypoallergenic material, such that a rash on a user's hand does not occur when the hand is restrained.

A wrist strap 15 is disposed on the upper surface 12, wherein the wrist strap 15 is configured to encircle a wrist and thereby removably secure the wrist to the padded base 11. In the illustrated embodiment, the wrist strap 15 is configured to selectively encircle only the wrist of a user, such that the hand of the user is unobscured, thereby allowing another individual, such as a nurse, to view the stability of an IV inserted into the hand. In one embodiment, the wrist strap 15 further comprises an internal padding 23 to reduce friction and bruising on a user's wrist when the wrist strap 15 is fastened. A peripheral padding 16 surrounds the padded base 11, extending outwardly from a perimeter of the upper surface 12. In this way, an open portion 17 is formed proximal to the upper surface 12. A secondary wrist strap 20 and mesh netting 18 are secured after the wrist strap 15 is secured.

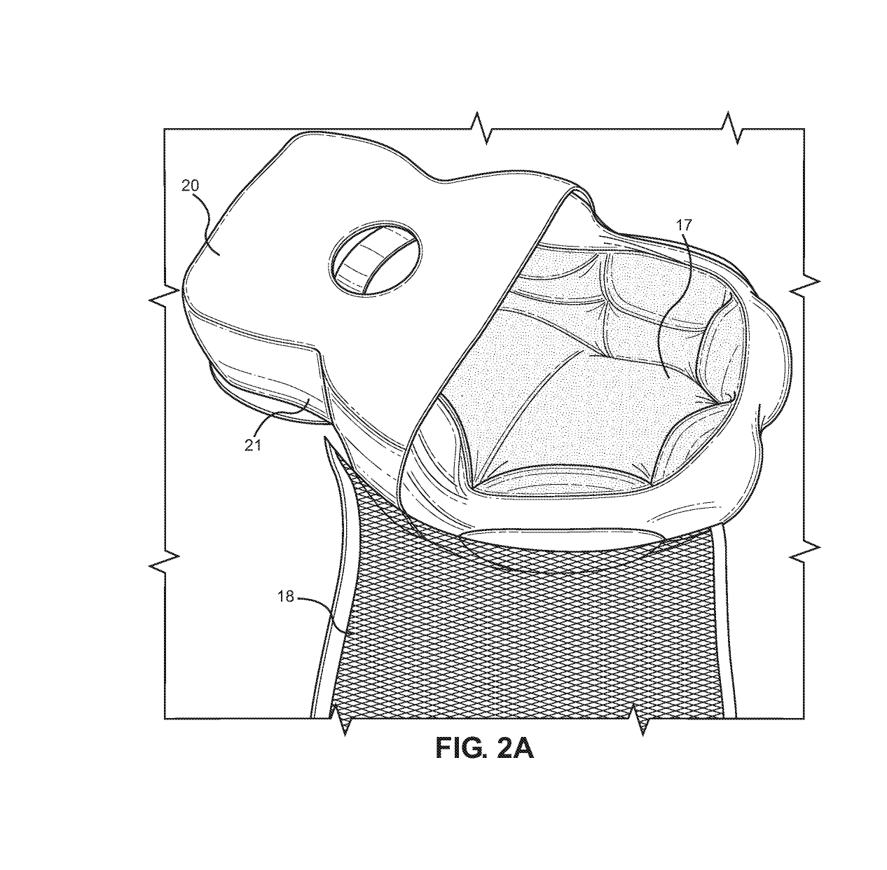

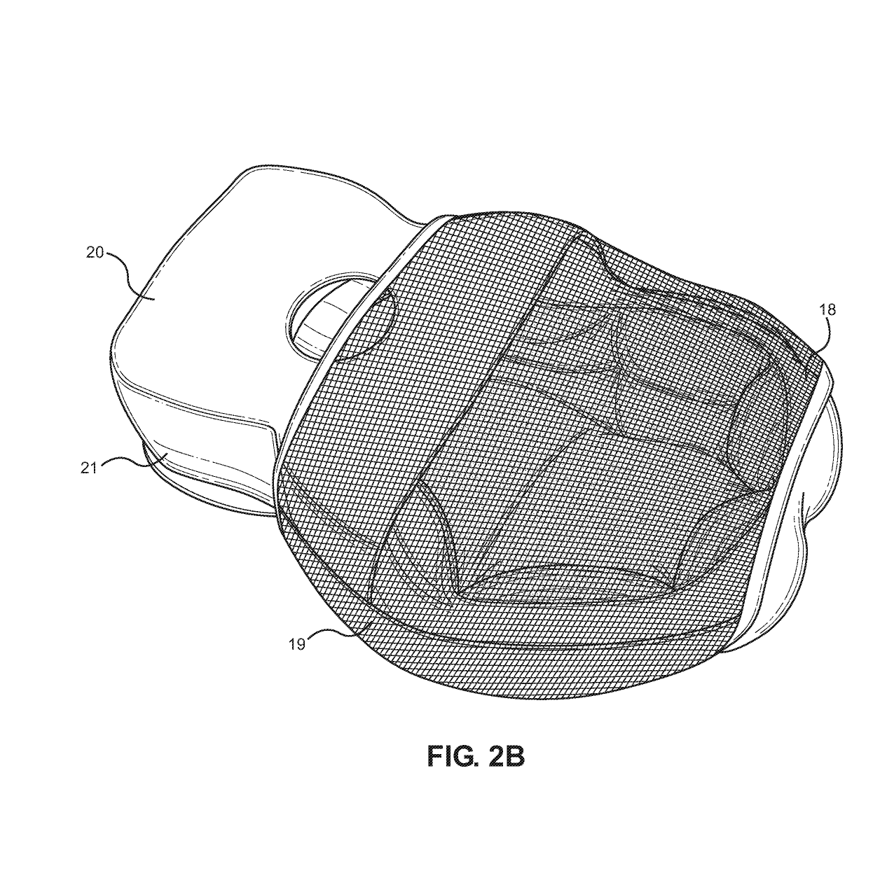

Referring now to FIGS. 2A and 2B, there are shown perspective views of an embodiment of the padded hand restraint with the secondary wrist strap fastened, and the secondary wrist strap and netting fastened, respectively. The secondary wrist strap 20 is affixed to a portion of the protrusion of the peripheral padding 16, wherein the secondary wrist strap 20 is configured to removably secure to the opposing portion of peripheral padding 16 via a fastener 21. In the illustrated embodiment, the fastener 21 is a hook and loop fastener, however in alternate embodiments any suitably secure fastener, such as snaps, can be used. In this way, the secondary wrist strap 20 is configured to further secure the wrist of the user and additionally secure the lower forearm of the user. A mesh netting 18 is affixed to an annular portion of the peripheral padding 16. The mesh netting 18 is configured to removably secure to the opposing portion of peripheral padding 16 via a fastener 19, such that the mesh netting is fastened overtop the open portion 17. In the illustrated embodiment, the fastener 19 comprises a hook and loop fastener. However, in additional embodiments the fastener 19 comprises other additional fasteners such as snaps.

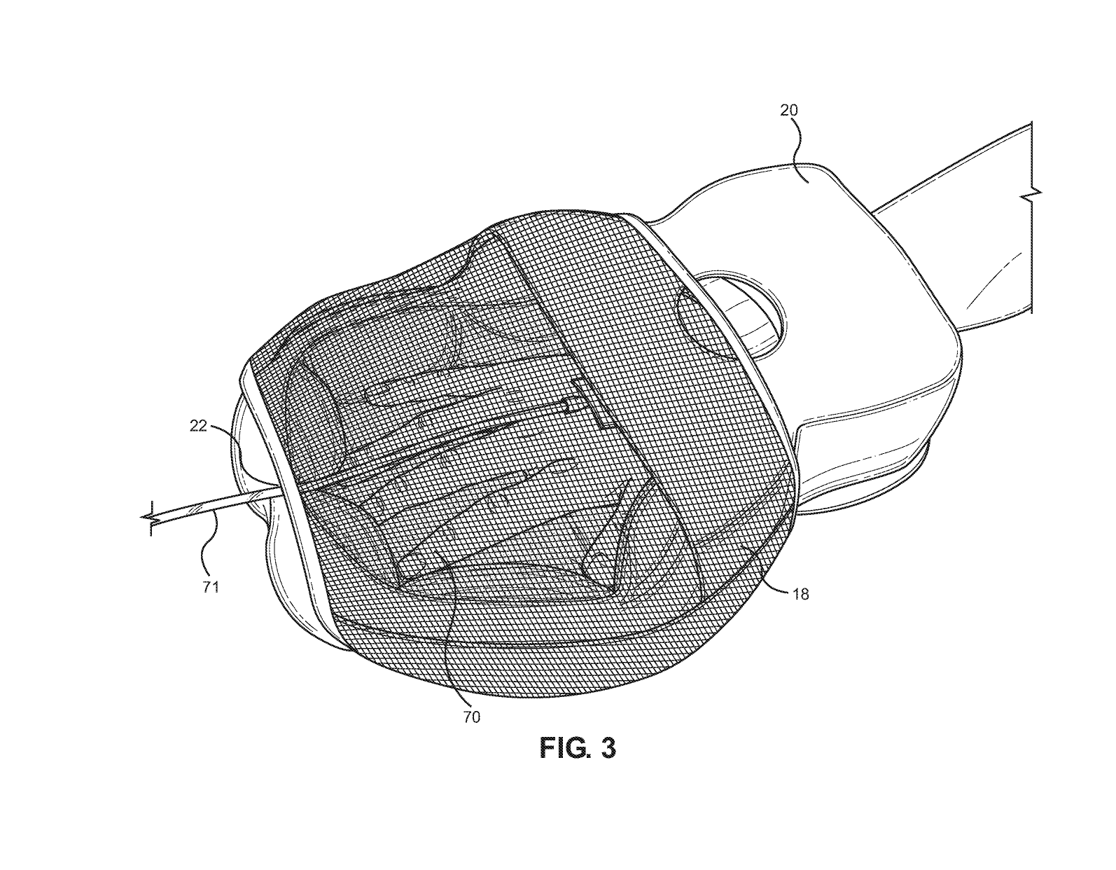

Referring now to FIG. 3, there is shown a perspective view of an embodiment of the padded hand restraint in use. The mesh netting 18 is configured to enclose a hand 70 within the open portion of the padded base 11. In this way, the hand 70 is prevented from reaching outside the padded base 11. However, the mesh netting 18 is configured to be semi-transparent wherein another individual, such as a nurse, could view the hand 70, thereby confirming an IV line 71 is still safely affixed to the hand 70. In an embodiment of the padded hand restraint, an aperture 22 is disposed at an apex of the annular portion of the peripheral padding 16, wherein the aperture 22 is configured to receive the IV line 71 therethrough. In other embodiments, the IV line 71 is disposed along an upper portion of the peripheral padding such that the mesh netting 18 is removably secured overtop the IV line 71.

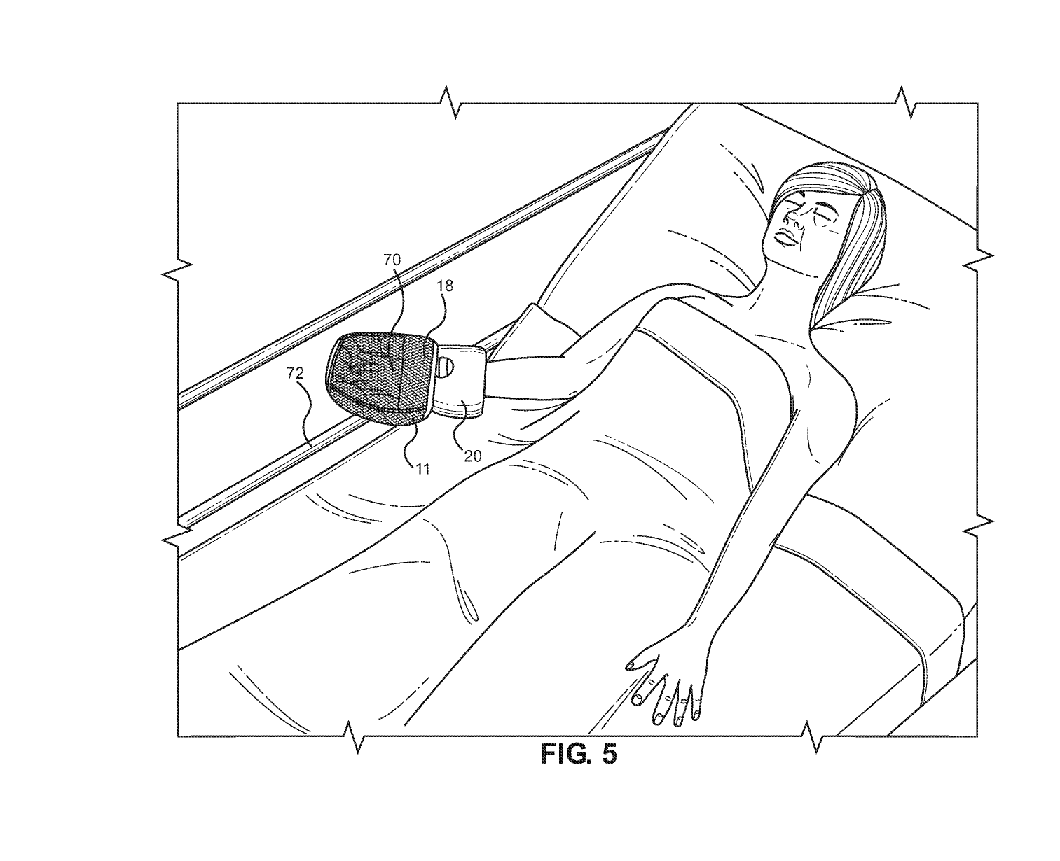

Referring now to FIG. 4 and FIG. 5, there is shown a perspective view of an embodiment of the padded hand restraint with the lower surface affixed to a bed frame, and an embodiment of the padded hand restraint in use affixed to a bed frame, respectively. A lower strap 14 is affixed to the lower surface 13. The lower strap 14 is configured to removably secure to a stationary object, such as a bed frame 72. In one embodiment, the lower strap 14 is configured to removably secure to the bed frame 72 via tying a knot. In another embodiment, the lower strap 14 is configured to removably secure to the bed frame 72 via a clip fastener. In this way, the padded hand restraint 11 can be removably secured to a stationary object, such as a bed frame 72, such that the padded hand restraint 11 cannot be removed by a user from the stationary object 72 once secured by another individual, such as a nurse. When a hand 70 is secured inside the padded hand restraint 11, such that the secondary wrist strap 20 and mesh netting 18 are both removably secured overtop the open portion, the hand 70 is safely restrained inside the padded hand restraint 11 such that a nurse can view the hand 70 without removing the padded hand restraint 11.

In operation, an individual, such as a nurse, will take a user, such as a patient, and place the patient's hand 70 into the open portion of the padded hand restraint, such that the wrist of the patient is disposed along the narrowed portion of the padded hand restraint. The padded hand restraint is padded with a cushion, such that the hand and wrist are not bruised or otherwise damaged when restrained therein. The wrist strap is removably fastened over the wrist of the patient, such that the hand is restrained within the padded hand restraint by the wrist. The secondary wrist strap 20 is then removably fastened over the wrist of the patient, such that the wrist and lower forearm of the patient are further restrained. The mesh netting 18 is removably fastened over the hand 70 of the patient, wherein the mesh netting 18 is transparent enough such that the hand and any IVs inserted into the hand are visible through and quickly accessible by the mesh netting. In one embodiment, an aperture disposed along the peripheral padding receives the IV therethrough. The lower strap 14 of the padded hand restraint is removably secured to a stationary object, such as a bed frame 72. In this way, a patient's hand or hands are restrained by a nurse such that the patient is unable to remove helpful medical equipment, such as an IV line, or otherwise harm themselves.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A padded hand restraint, comprising:
a padded base annularly shaped having a protrusion disposed along a rear portion of a perimeter such that a mitten shape is formed;
wherein the padded base has an upper surface and a lower surface, the lower surface having a lower strap thereon;
a wrist strap disposed on the upper surface, the wrist strap configured to secure a wrist to the padded base without obscuring a hand from view;
wherein a padding surrounds the base, the padding extending outwardly from a periphery of the upper surface, thereby defining an open portion configured to receive a hand therein;
a mesh netting disposed along a side portion of the peripheral padding and removably securable overtop the open portion via a fastener disposed on an opposite side portion of the peripheral padding;
a second strap disposed on a portion of peripheral padding configured to removably secure a hand to the padded base via a second fastener on an opposing peripheral side of the padded base.

2) The padded hand restraint of claim 1, wherein an aperture is disposed along the peripheral padding such that an IV line is receivable therethrough.

3) The padded hand restraint of claim 1, wherein the first wrist strap is padded along a lower surface.

4) The padded hand restraint of claim 1, wherein the upper surface is composed of a hypoallergenic material.

5) The padded hand restraint of claim 1, wherein the lower strap comprises a fastener configured to removably secure the padded hand restraint to a stationary object.
